(19) United States
(12) Patent Application Publication
(10) Pub. No.: US 2009/0175882 A1
(43) Pub. Date: Jul. 9, 2009

(54) METHODS AND COMPOSITIONS FOR TREATING HYPERALGESIA

(75) Inventors: Ardem Patapoutian, Del Mar, CA (US); Timothy J. Jegla, La Jolla, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignees: IRM LLC, Hamilton (BM); THE SCRIPPS RESEARCH INSTITUTE, La Jolla, CA (US)

(21) Appl. No.: 12/279,336

(22) PCT Filed: Feb. 21, 2007
(86) PCT No.: PCT/US2007/004640

§ 371 Date: Jan. 29, 2009

Related U.S. Application Data
(60) Provisional application No. 60/775,519, filed on Feb. 21, 2006.

(51) Int. Cl. A61K 39/395; A61K 31/485; A61K 31/405; A61K 31/195; A61K 31/16; A61K 31/15; C12Q 1/00

(52) U.S. Cl. 424/146.1; 514/640; 514/669; 514/282; 514/420; 514/568; 514/629; 435/4

(57) ABSTRACT

This invention provides compounds which specifically inhibit TRPA1 but not other members of the thermoTRP ion channel family. Also provided in the invention are methods of using TRPA1-specific inhibitors to treat or alleviate pains mediated by noxious mechanosensation.

### CROSS-REFERENCE TO RELATED APPLICATIONS

This patent application claims the benefit of priority under 35 U.S.C. §119(e) to U.S. Provisional Patent Application No. 60/775,519, filed Feb. 21, 2006. The disclosure of the priority application is incorporated herein by reference in its entirety and for all purposes.

### STATEMENT CONCERNING GOVERNMENT SUPPORT

This invention was made in part with government support under NINDS Grant Nos. NS42822 and NS046303 awarded by the National Institutes of Health. The U.S. Government may therefore have certain rights in this invention.

### FIELD OF THE INVENTION

The present invention generally relates to methods and compositions for antagonizing an ion channel involved in noxious chemosensation, thermosensation and mechanosensation. More particularly, the invention relates to compounds that specifically inhibit mechanotransduction mediated by TRPA1, and to methods of using such compounds to treat mechanical hyperalgesia.

### BACKGROUND OF THE INVENTION

Sensory neurons of the dorsal root ganglia (DRGs) can detect environmental changes through projections in the skin. Nociception is the process by which noxious stimuli such as heat and touch cause the sensory neurons (nociceptors) in the skin to send signals to the central nervous system. Some of these neurons are either mechanosensitive (high or low threshold) or thermosensitive (hot-, warm-, or cool-responsive). Still other neurons, called polymodal nociceptors, sense both noxious thermal (cold and hot) and mechanical stimuli.

Ion channels play a central role in neurobiology as membrane-spanning proteins that regulate the flux of ions. Categorized according to their mechanism of gating, ion channels can be activated by signals such as specific ligands, voltage, or mechanical force. A subset of the Transient Receptor Potential (TRP) family of cation channels dubbed thermoTRPs have been implicated in thermal sensation, e.g., TRPM8 and TRPA1. TRPM8 is activated at 25° C. It is also the receptor for the compound menthol, providing a molecular explanation of why mint flavors are typically perceived as refreshingly cooling. TRPA1, also termed ANKTM1, is activated at 17° C. It is an ion channel expressed in polymodal sensory neurons and can be activated by noxious cold and a variety of natural pungent compounds that cause a burning/pain sensation. See, e.g., Patapoutian et al., Nat. Rev. Neurosci. 4:529-539, 2003; Story et al., Cell 112: 819-829, 2003; and Bandell et al., Neuron. 41:849-57, 2004.

Mechanical sensation is inextricably linked to pain states in many diseases and medical conditions. For example, mechanotransduction is an important component of pain sensation associated with arthritis and neuropathic pain. However, as opposed to that for noxious thermal sensation, the molecular identity of mechanotransduction channels responsible for sensing noxious mechanical forces that are relevant for pain is unknown. The present invention addresses this and other unfulfilled needs in the art.

### SUMMARY OF THE INVENTION

In one aspect, the present invention provides methods for treating hyperalgesia in a subject. The methods involve administering to the subject a pharmaceutical composition that comprises an effective amount of a TRPA1 antagonist which, by specifically blocking TRPA1 activation, suppresses or inhibits noxious chemosensation, thermosensation, and mechanosensation in the subject. In some of the methods, the TRPA1 antagonist employed does not block activation of one or more of the other thermoTRPs selected from the group consisting of TRPV1, TRPV2, TRPV3, TRPV4 and TRPM8. In some methods, the TRPA1 antagonist used is (Z)-4-(4-chlorophynyl)-3-methylbut-3-en-2-oxime. In some other methods, the TRPA1 antagonist used is N,N′-Bis-(2-hydroxybenzyl)-2,5-diamino-2,5-dimethylhexane. In some other methods, a TRPA1 antagonist antibody is employed.

Some of the therapeutic methods of the invention are directed to treating subjects suffering from inflammatory conditions or neuropathic pains. In some of the methods, the subject being treated suffers from mechanical or thermal hyperalgesia. In some methods, the subject being treated is a human. In addition to the TRPA1 antagonist, a second pain-reducing agent is administered to the subject in some of the therapeutic methods. For example, the second pain-reducing agent can be an analgesic agent selected from the group consisting of acetaminophen, ibuprofen and indomethacin and opioids. The second pain-reducing agent can also be an analgesic agent selected from the group consisting of morphine and moxonidine.

In another aspect, the invention provides methods for identifying an agent that inhibits or suppresses noxious mechanosensation. These methods entail (a) contacting test compounds with a cell that expresses the transient receptor potential ion channel TRPA1, and (b) identifying a compound that inhibits a signaling activity of an activated TRPA1 in the cell in response to a mechanical stimulus. In some of these methods, the identified compound are further examined for effect on activation or signaling activities of one or more thermoTRPs selected from the group consisting of TRPV1, TRPV2, TRPV3, TRPV4 and TRPM8. In some methods, the identified compound suppresses or reduces the signaling activity of the activated TRPA1 ion channel relative to the signaling activity of the TRPA1 ion channel in the absence of the compound. In some of the methods, the identified compound does not block activation of one or more thermoTRPs selected from the group consisting of TRPV1, TRPV2, TRPV3, TRPV4 and TRPM8.

In some of these screening methods, the TRPA1 ion channel is activated by a TRPA1 agonist selected from the group consisting of cinnamaldehyde, eugenol, gingerol, methyl salicylate, and allicin. Examples of cells that can be employed in these methods include a TRPA1-expressing CHO cell, a TRPA1-expressing Xenopus oocyte, and a cultured DRG neuron. The signaling activity to be monitored in the methods can be, e.g., TRPA1-induced electric current across membrane of the cell or calcium influx into the cell. The mechanical stimulus applied in the screening can be, e.g., suction pressure or hyperosmotic stress.

The invention further provides a use of a TRPA1-specific inhibitor in the manufacture of a medicament for treating thermal or mechanical hyperalgesia in a subject. The TRPA1-specific inhibitors to be employed are, e.g., (Z)-4-(4-chlorophynyl)-3-methylbut-3-en-2-oxime or N,N′-Bis-(2-hydroxybenzyl)-2,5-diamino-2,5-dimethylhexane. Pharmaceutical compositions comprising these TRPA1-specific inhibitors are also provided in the invention.

A further understanding of the nature and advantages of the present invention may be realized by reference to the remaining portions of the specification and claims.

### DESCRIPTION OF THE DRAWINGS

FIGS. 1A-1D show that TRPA1 is activated by mechanical stimuli. (A) Currents recorded from TRPA1-expressing cells in response to cold (right, n=62), hypertonic osmolarity (middle, n=8), and (−) pressure (left, n=10) applied from the recording pipette; (B) Representative current-voltage relationship in response to different stimuli that activate TRPA1. (C) TRPA1 cells show robust current responses to negative pressures of −90 mmHg or higher. Values on the filled bars demonstrate number of responders out of all patches tested upon the relevant pressure. (D) A sub-threshold cold pre-pulse sensitizes the response of TRPA1 cells to a low threshold mechanical stimulus (n=5).

FIGS. 2A-2D show that TRPA1's mechano-responses are blocked by various known agents. (A) Gd3+ completely blocks current activation of TRPA1 upon hyperosmolarity (n=5 out of 5 cells) as does 5 μM ruthenium red (n=5 out of 5 cells for (−) pressure and n=6 out of 6 cells for hyperosmolarity). (B) A cinnamaldehyde-sensitive DRG neuron responds to −200 mmHg and to capsaicin. The current-voltage relationship in response to the negative pressure (collected from the location with an asterisk on the trace) is shown. (C) 2 mM camphor completely blocks current activation of TRPA1 upon (−) pressure in CHO cells (n=5). (D) 2 mM camphor completely blocks current response to (−) pressure of DRG neurons (n=15 out of 18 cells tested with (−) pressure). In 12 out of the 15 cells currents were also activated by 500 μM cinnamaldehyde.

FIGS. 3A-3D show that Compound 18 blocks TRPA1 activation. (A) Chemical structures of the compound 18 (upper) and cinnamaldehyde (lower). (B) Dose-response relationships for block of the calcium influx by compound 18 into CHO cells expressing mouse and human TRPA1 elicited by 50 μM cinnamaldehyde (left panel). Calcium influx was measured using a standard FLIPR assay, data points are the average of 4 wells (˜8,000 cells/well) and error bars show standard error. Values are normalized to the maximal response (observed in the absence of compound 18). IC₅₀ values are 3.1 μM and 4.5 μM for human and mouse, respectively. Compound 18 shifts the EC₅₀ of cinnamaldehyde on mouse TRPA1 rightward in a concentration-dependent manner (right panel). Data was generated using the FLIPR calcium-influx assay, n=3 wells (˜8,000 cells/well) and normalized to the maximal response. Bars show standard error and solid curves are hill equation fits from which EC₅₀ values are derived. EC₅₀ values for cinnamaldehyde are 50 μM (control), 111 μM (10 μM compound 18), and 220 μM (25 μM compound 18). Maximal responses were of similar magnitude in all cases. (C) Current-voltage relationship of TRPA1. Outward rectifying currents elicited by cinnamaldehyde (left panel) in inside-out macropatches of TRPA1-expressing Xenopus oocytes were suppressed by compound 18 coapplications (right panel). (D) Compound 18 suppresses acute nociceptive behaviours upon cinnamaldehyde but not capsaicin. Time spent licking and flicking hindpaws injected with cinnamaldehyde (16.4 mM) or capsaicin (0.328 mM) is measured for 5 min and compared with the hindpaw of another animal coinjected with compound 18 (1 mM). Numbers of cases for each experiment from the left is 8, 8, 6 and 6, respectively (***p<0.001, *p<0.05, two-tailed Student's T-test).

FIGS. 4A-4D show that TRPA1 mediates mechanical and cold hypersensitivity under inflammation (A-B). A novel TRPA1 blocker, compound 18, reverses CFA- (n=8) or BK-induced (n=12) nociceptive mechanical behaviours, but not thermal (heat) behaviours (n=8 for each of CFA and BK) in mice. Red symbols represent responses from CFA-injected (A), or BK-injected (B) hindpaws while blue symbols represent responses from the other noninjected hindpaws of the same animals. Circles represent responses upon compound 18 treatment, whereas triangles represent responses upon vehicle treatments (A-C). Von Frey thresholds are measured and averaged. (***p<0.001, *p<0.05, two-tailed Student's T-test). (C) Compound 18 reverses cold behaviours of rats injected with CFA. Red symbols represent responses from CFA-injected hindpaws while blue symbols represent responses from the other non-injected hindpaws of the same animals. Number of flicks, licks, paw raises for 10 min at each time point are counted and averaged (n=8, *p<0.05, two-tailed Student's T-test). (D) 1 nM BK pre-pulse sensitizes the response of TRPA1 CHO cells coexpressing B2 receptor to a low threshold mechanical stimulus. 2 mM camphor was incubated during the BK pulse to protect mild activation and subsequent desensitization of TRPA1 by BK. The results indicate that mechanical threshold of the cells was shifted down to −60 mmHg.

### DETAILED DESCRIPTION

### I. Overview

The present invention is predicated in part on the findings by the present inventors that TRPA1, in addition to being an important component of pain sensation that signals noxious cold temperature, is also a sensor for noxious mechanical stimuli. The inventors also identified compounds that specifically inhibit activation of TRPA1, but not other ion channels of the Trp family. As detailed in the Examples below, the present inventors discovered that TRPA1 is activated by noxious mechanical forces, and that this activation is facilitated under inflammatory conditions. It was further discovered that small molecule inhibitors of TRPA1 can significantly reduce nociceptive behavior in response to cinnamaldehyde but not capsaicin in mice. Furthermore, the inhibitors block mechanical and cold hyperalgesia, but not heat hyperalgesia.

In accordance with these discoveries, the invention provides methods of screening for therapeutic agents that can be used to suppress or inhibit noxious mechanosensation. Also provided in the invention are methods of employing TRPA1-specific inhibitors to alleviate pains associated with noxious mechanical stimuli in various diseases and conditions. The following sections provide guidance for making and using the compositions of the invention, and for carrying out the methods of the invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY (1991). In addition, the following definitions are provided to assist the reader in the practice of the invention.

The term “agent” or “test agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved traits (such as higher binding affinity for a target molecule) is an approach that is well known in pharmaceutical chemistry.

As used herein, “contacting” has its normal meaning and refers to combining two or more agents (e.g., polypeptides or small molecule compounds) or combining agents and cells. Contacting can occur in vitro, e.g., combining two or more agents or combining a test agent and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate.

As used herein, “hyperalgesia” or a “hyperalgesic state” refers to a condition in which a warm-blooded animal is extremely sensitive to mechanical, chemical or thermal stimulation that, absent the condition, would be painless. Hyperalgesia is known to accompany certain physical injuries to the body, for example the injury inevitably caused by surgery. Hyperalgesia is also known to accompany certain inflammatory conditions in man such as arthritic and rheumatic disease. Hyperalgesia thus refers to mild to moderate pain to severe pain such as the pain associated with, but not limited to, inflammatory conditions (e.g., such as rheumatoid arthritis and osteoarthritis), postoperative pain, post-partum pain, the pain associated with dental conditions (e.g., dental caries and gingivitis), the pain associated with burns, including but not limited to sunburns, abrasions, contusions and the like, the pain associated with sports injuries and sprains, inflammatory skin conditions, including but not limited to poison ivy, and allergic rashes and dermatitis, and other pains that increase sensitivity to mild stimuli, such as noxious cold.

The term “modulate” with respect to a reference protein (e.g., a TRPA1) refers to inhibition or activation of a biological activity of the reference protein (e.g., a pain signaling related activity of TRPA1). Modulation can be up-regulation (i.e., activation or stimulation) or down-regulation (i.e., inhibition or suppression). The mode of action can be direct, e.g., through binding to the reference protein as a ligand. The modulation can also be indirect, e.g., through binding to and/or modifying another molecule which otherwise binds to and modulates the reference protein.

“Neuropathic pain” encompasses pain arising from conditions or events that result in nerve damage. “Neuropathy” refers to a disease process resulting in damage to nerves. “Causalgia” denotes a state of chronic pain following nerve injury or a condition or event, such are cardiac infarction, that causes referred pain. “Allodynia” comprises a condition in which a person experiences pain in response to a normally nonpainful stimulus, such as a gentle touch. An “analgesic agent” is a molecule or combination of molecules that causes a reduction in pain. An analgesic agent employs a mechanism of action other than inhibition of TRPA1 when its mechanism of action does not involve direct (via electrostatic or chemical interactions) binding to and reduction in the function of TRPA1.

“Polynucleotide” or “nucleic acid sequence” refers to a polymeric form of nucleotides (polyribonucleotide or polydeoxyribonucleotide). In some instances a polynucleotide refers to a sequence that is not immediately contiguous with either of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. Polynucleotides can be ribonucleotides, deoxyribonucleotides, or modified forms of either nucleotide.

A polypeptide or protein (e.g., TRPA1) refers to a polymer in which the monomers are amino acid residues that are joined together through amide bonds. When the amino acids are alpha-amino acids, either the L-optical isomer or the D-optical isomer can be used, the L-isomers being typical. A polypeptide or protein fragment (e.g., of TRPA1) can have the same or substantially identical amino acid sequence as the naturally occurring protein. A polypeptide or peptide having substantially identical sequence means that an amino acid sequence is largely, but not entirely, the same, but retains a functional activity of the sequence to which it is related.

Polypeptides may be substantially related due to conservative substitutions, e.g., TRPA1 and a TRPA1 variant containing such substitutions. A conservative variation denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic for aspartic acids, or glutamine for asparagine, and the like. Other illustrative examples of conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine to leucine.

The term “subject” includes mammals, especially humans, as well as other non-human animals, e.g., horse, dogs and cats.

A “variant” of a reference molecule (e.g., a TRPA1 polypeptide or a TRPA1 modulator) is meant to refer to a molecule substantially similar in structure and biological activity to either the entire reference molecule, or to a fragment thereof. Thus, provided that two molecules possess a similar activity, they are considered variants as that term is used herein even if the composition or secondary, tertiary, or quaternary structure of one of the molecules is not identical to that found in the other, or if the sequence of amino acid residues is not identical.

### III. TRPA1 Specific Inhibitors

Since TRPA1 is a receptor for noxious chemical, thermal and mechanical stimuli, TRPA1 antagonist compounds are useful in reducing pain associated with somatosensation, including mechanosensation, e.g., mechanical hyperalgesia and allodynia. Compounds that specifically inhibit or suppress mechanosensation mediated by TRPA1 can have various therapeutic or prophylactic (e.g., antinociceptive) applications. Any molecule that inhibits the TRPA1 ion channel might be able to lessen pain mediated by noxious stimuli such as mechanosensation. However, molecules which are capable of inhibiting other thermoTRPs (e.g., TRPV1, TRPV2, TRPV3 and TRPM8) in addition to TRPA1 can interfere with the various functions performed by those molecules. Such nonselective inhibitors of TRPA1, although being able to diminish pain, are likely to have many unwanted side effects. Thus, molecules that selectively inhibit the TRPA1 ion channel are preferred in such therapeutic applications. By specifically inhibiting TRPA1 mediated signaling while causing no effect on signaling of the other thermoTRPs, symptoms of a subject suffering from mechanical hyperesthesia can be reduced or inhibited.

TRPA1 inhibitors that can be employed in the practice of the present invention include compounds that interferes with the expression, modification, regulation or activation of TRPA1, or compounds that down-regulates one or more of the normal biological activities of TRPA1 (e.g., its ion channel). A selective inhibitor of TRPA1 significantly blocks TRPA1 activation or inhibits TRPA1 signaling activities at a concentration at which activation or signaling activities of the other thermalTRPs (e.g., TRPV1, TRPV2, TRPV3, TRPV4 and or TRPM8) are not significantly affected. Various TRPA1-specific antagonists can be used in the instant invention. Some of these TRPA1-specific inhibitors are identified by the present inventors, as described in the Examples below. These compounds can be obtained commercially or are otherwise described in the art. One such compound is Compound 18, (Z)-4-(4-chlorophynyl)-3-methylbut-3-en-2-oxime. This compound can be obtained commercially from Maybridge (Cornwall, UK). Another example is Compound 40, N,N′-Bis-(2-hydroxybenzyl)-2,5-diamino-2,5-dimethylhexane, which has been described in U.S. Pat. No. 4,129,556. As shown in the Examples below, these two compounds are able to specifically inhibit TRPA1 activation or function, and thereby suppress TRPA1-mediated mechanical norciception. They have little or no effect on activation or activities of the other thermTRPs such as TRPV1, TRPV2, TRPV3, TRPV4, or TRPM8. Thus, these two compounds can be readily used to treat or alleviate mechanical hyperalgesia as described in more detail below.

Other than these exemplified TRPA1-specific antagonists, additional TRPA1-specific inhibitors can be readily identified using methods described herein or methods that have been described in the art. Novel TRPA1 antagonists that can be identified with these screening methods include small molecule organic compounds and antagonist antibodies that specifically inhibit TRPA1 activity in sensing mechanical stimuli. Antagonist antibodies of TRPA1, preferably monoclonal antibodies, can be generated using methods well known in the art. For example, the production of non-human monoclonal antibodies, e.g., murine or rat, can be accomplished by, for example, immunizing the animal with a TRPA1 polypeptide or its fragment (See Harlow & Lane, Antibodies, A Laboratory Manual, Cold Spring Harbor Laboratory Press, New York, 1988). Such an immunogen can be obtained from a natural source, by peptides synthesis or by recombinant expression.

Novel small molecule TRPA1 can be identified by screening test compounds for ability to inhibit TRPA1 ion channel activities. To screen for compounds that antagonize the signaling activities of TRPA1, TRPA1 must be activated first. One way to accomplish this is to apply cold. However, this approach is not practical in a high throughout screening format. In the methods described in PCT Application WO05/089206, a TRPA1 agonist compound such as bradykinin, eugenol, gingerol, methyl salicylate, allicin, and cinnamaldehyde is used to activate TRPA1. Test compounds can then be screened for ability to block activation of TRPA1 by any of these TRPA1 agonists or inhibit signaling activities of an activated TRPA1 ion channel.

By way of example, the screening methods of the present invention typically involve contacting a TRPA1-expressing cell with test compounds, and identifying a compound that suppresses or inhibits a biological or signaling activity of the activated TRPA1 in the cell in response to a mechanical stimulus. TRPA1 in the cell can be activated by the addition one of the above noted TRPA1 agonist compounds before, concurrently with, or after contacting the cell with test compounds. The compounds can be screened for ability to modulate calcium influx or intracellular free calcium level of a TRPA1-expressing cell or a cultured DRG neuron in response to mechanical stimuli. As described in the Examples herein, modulating effect of test compounds on TRPA1-mediated mechanosensation can be examined by the FLIPR assay using TRPA1-expressing CHO cells or cultured rat DRGs in response to a mechanical pressure (e.g., suction) or hyperosmotic stress. They can also be assayed for activity in modulating whole-cell membrane currents of TRPA1-expressing cells, e.g., by recording cinnamaldehyde-induced TRPA1 currents in excised patches of Xenopus oocytes. Preferably, these screening methods are performed in a high throughput format. For example, each test compound can be put into contact with a TRPA1-expressing cell in a different well of a microtiter plate. The TRPA1 agonist is present in each of these wells to activate TRPA1.

If a test compound suppresses or inhibits the activity of the activated TRPA1 (e.g., an ion channel activity), a candidate TRPA1 antagonist or inhibitor is identified. As a control, the candidate TRPA1 antagonist is also tested for any effect on the signaling or ion channel activities of one or more of the other thermoTRP channels, as illustrated in the Examples below. This allows identification of TRPA1-specific inhibitors that would not affect the normal functions of the other thermoTRP channels. In some embodiments, the identified TRPA1-specific antagonist can be further examined in suitable animal models in vivo, e.g., by the behavioral assays (paw withdrawal assay) with rats or mice as disclosed in the Examples below. Additional guidance for performing hyperalgesia assays has been described in the literature, e.g., Morqrich et al., Science 307:1468, 2005; and Caterina et al., Science 288:306, 2000. As control, similar animal models can also be employed to ascertain that the candidate TRPA1-specific antagonists do not have any significant effect on the other thermoTRPs in vivo.

Test compounds that can be screened for novel TRPA1 modulators (e.g., inhibitors) include polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, polynucleotides (e.g., inhibitory nucleic acids such as siRNAs) polypeptides, saccharides, fatty acids, steroids, purines, pyrimidines, derivatives, structural analogs or combinations thereof. Some test agents are synthetic molecules, and others natural molecules. In some preferred methods, the test agents are small organic molecules (e.g., molecules with a molecular weight of not more than about 500 or 1,000). Preferably, high throughput assays are adapted and used to screen for such small molecules. In some methods, combinatorial libraries of small molecule test agents can be readily employed to screen for small molecule modulators of TRPA1. A number of assays known in the art can be readily modified or adapted in the practice of the screening methods of the present invention, e.g., as described in Schultz et al., Bioorg Med Chem Lett 8: 2409-2414, 1998; Weller et al., Mol Divers. 3: 61-70, 1997; Fernandes et al., Curr Opin Chem Biol 2: 597-603, 1998; and Sittampalam et al., Curr Opin Chem Biol 1: 384-91, 1997.

### IV. Treating Mechanical Hyperalgelsia with TRPA1-Specific Inhibitors

The invention provides methods for reducing pain sensation under physiological and pathophysiological conditions (e.g., allodynia and hyperalgesia), especially pain perception that is associated with or mediated by mechanosensation through TRPA1. For example, mechanical hyperalgesia is present in many medical disorders. For example, inflammation can induce hyperalgesia. Examples of inflammatory conditions include osteoarthritis, colitis, carditis, dermatitis, myositis, neuritis, collagen vascular diseases such as rheumatoid arthritis and lupus. Subjects with any of these conditions often experience enhanced sensations of pain of which mechanical hyperalgesia is a component. Other medical conditions or procedures that may cause excessive pain include trauma, surgery, amputation, abscess, causalgia, demyelinating diseases, trigeminal neuralgia, chronic alcoholism, stroke, thalamic pain syndrome, diabetes, cancer viral infections, and chemotherapy. Mechanosensation can play an important role in the norciception of any of these conditions.

Typically, the methods involve administering to a subject in need of treatment a pharmaceutical composition that contains a TRPA1-specific inhibitor of the present invention. The TRPA1-specific inhibitor can be used alone or in conjunction with other known analgesic agents to alleviate pain in a subject. Examples of such known analgesic agents include morphine and moxonidine (U.S. Pat. No. 6,117,879). Subjects that are suitable for treatment with the methods of the invention are those who are suffering from mechanical hyperesthesia (hyperalgesia in particular) or those who have a medical condition or disorder in which noxious mechanosensation plays a role. They include human subjects, non-human mammals and other subjects or organisms that express TRPA1. The subjects may have an ongoing condition that is currently causing pain and is likely to continue to cause pain. They may also have been or will be enduring a procedure or event that usually has painful consequences. For example, the subject may have chronic painful conditions such as diabetic neuropathic hyperalgesia or collagen vascular diseases. The subject may also have inflammation, nerve damage, or toxin exposure (including exposure to chemotherapeutic agents). The treatment or intervention is intended to reducing or lessening pain in a subject so that the level of pain the subject perceives is reduced relative to the level of pain the subject would have perceived were it not for the treatment.

Generally, the treatment should affect a subject, tissue or cell to obtain a desired pharmacologic and/or physiologic effect. The effect may be prophylactic in terms of completely or partially preventing a disease or sign or symptom thereof. It can also be therapeutic in terms of a partial or complete cure for hyperalgesia and nociceptive pain associated disorders and/or adverse effect (e.g., pain) that is attributable to the disorders. Where the subject is a human, the level of pain the person perceives can be assessed by asking him or her to describe the pain or compare it to other painful experiences. Alternatively, pain levels can be calibrated by measuring the subject's physical responses to the pain, such as the release of stress-related factors or the activity of pain-transducing nerves in the peripheral nervous system or the CNS. One can also calibrate pain levels by measuring the amount of a well characterized analgesic required for a person to report that no pain is present or for a subject to stop exhibiting symptoms of pain.

Preferably, the methods are directed to alleviating either acute or chronic pain which has a mechanical hyperalgelsia component. The difference between “acute” and “chronic” pain is one of timing: acute pain is experienced soon (preferably within about 48 hours, more preferably within about 24 hours, most preferably within about 12 hours) after the occurrence of the event (such as inflammation or nerve injury) that led to such pain. By contrast, there is a significant time lag between the experience of chronic pain and the occurrence of the event that led to such pain. Such time lag is at least about 48 hours after such event, preferably at least about 96 hours after such event, more preferably at least about one week after such event. In some embodiments of the invention, a TRPA1-specific inhibitor is used to treat a subject suffering from an inflammatory pain. Such inflammatory pain may be acute or chronic and can be due to any number of conditions characterized by inflammation including, without limitation, sunburn, rheumatoid arthritis, osteoarthris, colitis, carditis, dermatitis, myositis, neuritis and collagen vascular diseases.

In some other embodiments, treatment of subjects having neuropathic pain is intended. These subjects can have a neuropathy classified as a radiculopathy, mononeuropathy, mononeuropathy multiplex, polyneuropathy or plexopathy. Diseases in these classes can be caused by a variety of nerve-damaging conditions or procedures, including, without limitation, trauma, stroke, demyelinating diseases, abscess, surgery, amputation, inflammatory diseases of the nerves, causalgia, diabetes, collagen vascular diseases, trigeminal neuralgia, rheumatoid arthritis, toxins, cancer (which can cause direct or remote (e.g. paraneoplastic) nerve damage), chronic alcoholism, herpes infection, AIDS, and chemotherapy. Nerve damage causing hyperalgesia can be in peripheral or CNS nerves. This embodiment of the invention is based on experiments showing that administration of a TRPA1 inhibitor significantly diminishes hyperalgesia due to diabetes, chemotherapy or traumatic nerve injury.

In some embodiments of the invention, subjects in need of treatment or alleviation of mechanical hyperalgelsia are administered with a composition combining an inhibitor of TRPA1 with one or more additional pain-reducing agents. This is because an individual pain medication often provides only partially effective pain alleviation because it interferes with just one pain-transducing pathway out of many. However, pain associated with diseases or medical conditions often involves multiple norciceptors and different signaling pathways, e.g., both mechanosensation and thermosensation. Thus, more than one pain-reducing agent is usually needed to alleviate norciception in these situations. In some other applications, TRPA1 inhibitors can be administered in combination with an analgesic agent that acts at a different point in the pain perception process. For example, one class of analgesics, such as NSAIDs (e.g., acetaminophen, ibuprofen and indomethacin), down-regulates the chemical messengers of the stimuli that are detected by the nociceptors. Another class of drugs, such as opioids, alters the processing of nociceptive information in the CNS. Other analgesics such as local anesthetics including anticonvulsants and antidepressants can also be included. Administering one or more classes of drug in addition to TRPA1 inhibitors can provide more effective amelioration of pain.

### V. Pharmaceutical Compositions and Administration

Subjects in need of treatment or alleviation of pain mediated by noxious mechanosensation can be administered with a TRPA1-specific inhibiting compound alone. However, the administration of a pharmaceutical composition that contains the TRPA1-specific inhibitor is more preferred. Examples of TRPA1-specific inhibitors that can be employed in the pharmaceutical compositions include Compound 18 or Compound 40 described in the Examples below. Novel TRPA1 inhibitors that can be identified in accordance with the screening methods of the invention can also be used. The invention also provides for a pharmaceutical combination, e.g. a kit. Such pharmaceutical combination can contain an active agent which is a TRPA1-inhibiting compound disclosed herein, in free form or in a composition, at least one co-agent, as well as instructions for administration of the agents.

The pharmaceutical compositions that comprise a TRPA1 inhibiting compound can be prepared in various forms. Suitable solid or liquid pharmaceutical preparation forms are, e.g., granules, powders, tablets, coated tablets, (micro)capsules, suppositories, syrups, emulsions, suspensions, creams, aerosols, drops or injectable solution in ampule form and also preparations with protracted release of active compounds. They can be prepared in accordance with the standard protocols well known in the art, e.g., Remington: The Science and Practice of Pharmacy, Gennaro, ed., Lippincott Williams & Wilkins (20^{th} ed., 2003). The pharmaceutical compositions typically contain an effective amount of the TRPA1 inhibiting compound that is sufficient to lessen or ameliorate pain associated with or mediated by TRPA1. In addition to the TRPA1-inhibiting compounds, the pharmaceutical compositions can also contain certain carriers which enhance or stabilize the composition, or facilitate preparation of the composition. For example, the TRPA1-inhibiting compound can be complexed with carrier proteins such as ovalbumin or serum albumin prior to their administration in order to enhance stability or pharmacological properties. The various forms of pharmaceutical compositions can also contain excipients and additives and/or auxiliaries such as disintegrants, binders, coating agents, swelling agents, lubricants, flavorings, sweeteners and elixirs containing inert diluents commonly used in the art, such as purified water.

Pharmaceutically acceptable carriers are determined in part by the particular composition being administered as well as by the particular method used to administer the composition. They should also be both pharmaceutically and physiologically acceptable in the sense of being compatible with the other ingredients and not injurious to the subject. The carrier may take a wide variety of forms depending on the form of preparation desired for administration, e.g., oral, sublingual, rectal, nasal, intravenous, or parenteral. For example, examples of non-aqueous solvents are propylene glycol, polyethylene glycol, vegetable oils such as olive oil, and injectable organic esters such as ethyl oleate. Carriers for occlusive dressings can be used to increase skin permeability and enhance antigen absorption. Liquid dosage forms for oral administration may generally comprise a liposome solution containing the liquid dosage form.

Pharmaceutical composition containing a TRPA1-inhibiting compound can be administered locally or systemically in a therapeutically effective amount or dose. They can be administered parenterally, enterically, by injection, rapid infusion, nasopharyngeal absorption, dermal absorption, rectally and orally. An effective amount means an amount that that is sufficient to reduce or inhibit a nociceptive pain or a nociceptive response in a subject. Such effective amount will vary from subject to subject depending on the subject's normal sensitivity to pain, its height, weight, age, and health, the source of the pain, the mode of administering the inhibitor of TRPA1, the particular inhibitor administered, and other factors. As a result, it is advisable to empirically determine an effective amount for a particular subject under a particular set of circumstances.

For a given TRPA1-inhibitor compound, one skilled in the art can easily identify the effective amount of an agent that modulates a nociceptive response by using routinely practiced pharmaceutical methods. Typically, dosages used in vitro may provide useful guidance in the amounts useful for in situ administration of the pharmaceutical composition, and animal models may be used to determine effective dosages for treatment of particular disorders. More often, a suitable therapeutic dose can be determined by clinical studies on mammalian species to determine maximum tolerable dose and on normal human subjects to determine safe dosage. Except under certain circumstances when higher dosages may be required, the preferred dosage of a TRPA1-specific inhibitor usually lies within the range of from about 0.001 to about 1000 mg, more usually from about 0.01 to about 500 mg per day. As a general rule, the quantity of a TRPA1-specific inhibitor administered is the smallest dosage which effectively and reliably prevents or minimizes the conditions of the subjects. Therefore, the above dosage ranges are intended to provide general guidance and support for the teachings herein, but are not intended to limit the scope of the invention. Additional guidance for preparation and administration of the pharmaceutical compositions of the invention has also been described in the art. See, e.g., Goodman & Gilman's The Pharmacological Bases of Therapeutics, Hardman et al., eds., McGraw-Hill Professional (10^{th} ed., 2001); Remington: The Science and Practice of Pharmacy, Gennaro, ed., Lippincott Williams & Wilkins (20^{th} ed., 2003); and Pharmaceutical Dosage Forms and Drug Delivery Systems, Ansel et al. (eds.), Lippincott Williams & Wilkins (7^{th} ed., 1999).

### EXAMPLES

The following examples are offered to illustrate, but not to limit the present invention.

### Example 1

### TRPA1 is a Polymodal Sensor of Noxious Mechanical and Thermal Stimuli

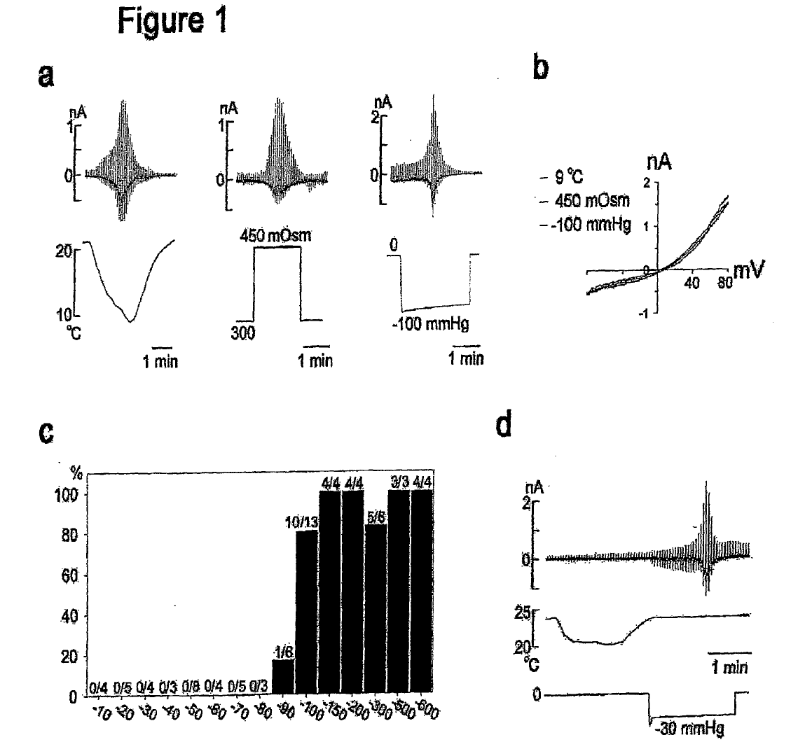

We tested if TRPA1 is activated by mechanical forces. The electrophysiological behavior of thermoTRP-expressing Chinese Hamster Ovary (CHO) cells was investigated with two different assays of mechanical stress—pressure application using the recording pipette and changes in external osmolarity. In whole cell recordings, TRPA1-expressing cells showed robust current responses to stimuli that cause cell shrinking (either −100 mmHg of suction (n=10) or application of a hyperosmotic 450 mOsm solution (n=8)) (FIG. 1A), but not cell swelling [either by +100 mmHg (n=11) or 220 mOsm (n=8). The currents evoked by pressure, hypertonicity, and cold (n=62) showed a similar desensitization, and have similar reversal potentials and rectification properties, suggesting that these mechanosensitive currents are due to TRPA1 activation (FIG. 11B). It was also observed that untransfected control CHO cells and other thermoTRPs (TRPV1, TRPV2, TRPV3, TRPM8) expressed in CHO cells did not respond to mechanical stimuli (data not shown), confirming that the TRPA1 response is specific.

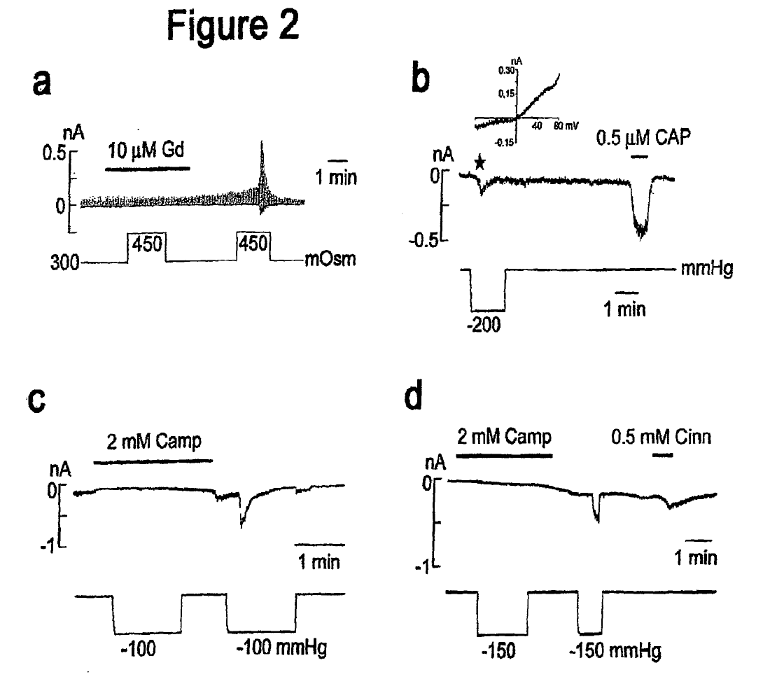

It is known that TRPV4 and other Drosophila TRPV family members respond to hypotonic solutions, and that TRPV4 knockout studies show that this channel is required for normal tail pressure responses. Mechanosensory neurons are often classified as high- or low-threshold, characterizing responses to pain and touch, respectively. We tested the mechanical threshold of TRPA1 by applying a wide range of negative pipette pressures (FIG. 1C). TRPA1-expressing CHO cells are activated at −90 mmHg or higher, consistent with native high-threshold mechanical receptors involved in sensing pain (Cho et al., J Neurosci 22:1238, 2002). Interestingly, a 20° C. cold pre-pulse sensitizes the TRPA1 response to low mechanical threshold of −30 mmHg (n=5), demonstrating that the activation threshold of TRPA1 can be modulated (FIG. 1D). We observed that 5 μM ruthenium red, a known TRPA1 blocker, completely blocked the mechanosensitive currents (for -100 mmHg, n=8; for 450 mOsm, n=5, data not shown), consistent with the mechanical responses originating from TRPA1. Gadolinium (Gd³⁺) is considered a blocker of native mechanically-gated ion channels in animal tissues (Martinac et al., Physiol Rev 81:685, 2001). We found that bath application of 10 μM Gd³⁺ completely and reversibly blocked TRPA1 currents in response to a 2 min-stimulus of 450 mOsm (n=5) (FIG. 2A) or -100 mmHg (n=6). FM1-43 is a styryl dye that specifically labels sensory cells by entering through open transduction channels. We found that FM1-43 treatment labeled CHO cells transfected with TRPA1 and treated with cinnamaldehyde. By contrast, TRPA1-expressing cells that were not activated by cinnamaldehyde did not take up the dye (data not shown). Furthermore, it was observed that 10 μM of FM1 -43 was able to block cinnamaldehyde-induced currents in TRPA1-expressing CHO cells (n=8). These results are consistent with TRPA1 being a sensory transduction channel.

To ensure that the mechanical response we observe is not an artifact of heterologous expression system, we tested if native TRPA1-expressing neurons also respond to such stimuli. 5/6 cinnamaldehyde-sensitive (presumptive TRPA1-expressing) DRG neurons responded to an application of −200 mmHg of suction, while 0/21 cinnamaldehyde insensitive neurons responded (16 of 21 were capsaicin-sensitive) (FIG. 2B). Millimolar camphor was recently reported to inhibit basal currents and cold or agonist-activation of TRPA1. We found that 2 mM camphor was also able to completely block mechanical response of TRPA1 in CHO cells to −100 mmHg (n=5, FIG. 2C). Consistently, currents of DRG neurons in response to −150 mmHg were fully inhibited by application of camphor at the same concentration (FIG. 2D) (n=15, 12 of 15 were cinnamaldehyde-sensitive). This data strongly supports that native TRPA1-expressing DRG neurons are mechanosensitive, displaying comparable characteristics to mechanosensitivity of TRPA1 in CHO cells.

### Example 2

### TRPA1 Plays an Essential Role in Mechanical Pain Sensation In Vivo

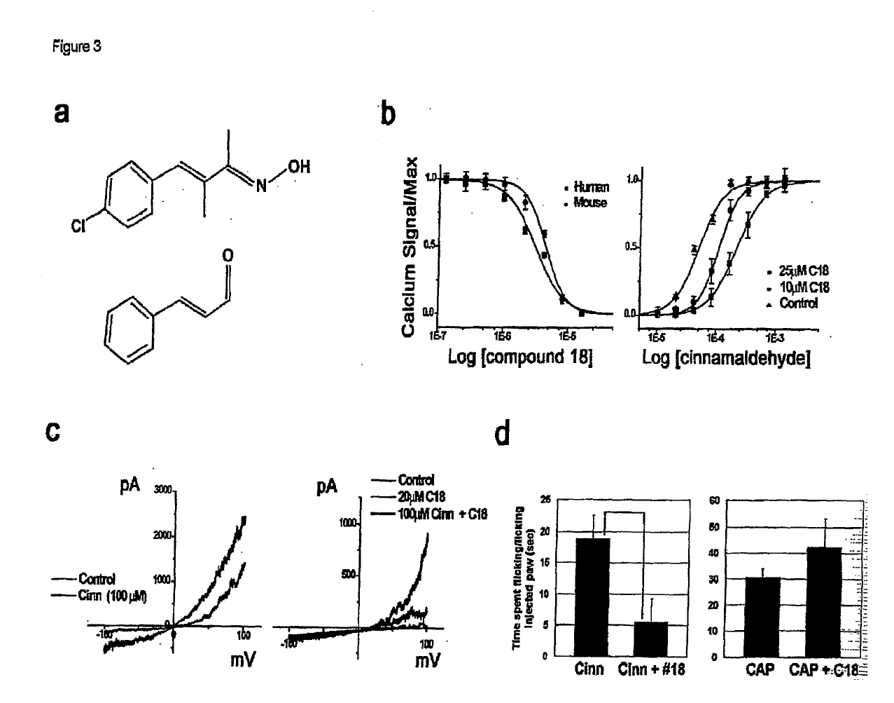

We next set out to test if acute block of TRPA1 had any physiological consequence on pain sensation. RR, Gd³⁺, or camphor are not specific compounds and can not be used in vivo. Using FLIPR calcium-influx assay, we screened 43,648 small molecules for their ability to block cinnamaldehyde-activation of human TRPA1 in a CHO cell line. Several hits appeared to be structural analogs of cinnamaldehyde. We performed in-depth analysis on one of these analogs, Compound 18, (Z)-4-(4-chlorophynyl)-3-methylbut-3-en-2-oxime (Maybridge, Cornwall, UK). Compound 18 blocked activation of TRPA1 by 50 μM cinnamaldehyde in the CHO cell FLIPR assay with an IC₅₀ of 3.1 μM and 4.5 μM for human and mouse clones, respectively (FIG. 3B). In contrast, it did not block TRPV1, TRPV3, TRPV4, and TRPM8 at 50 μM (data not shown). Compound 18 shifted the EC₅₀ for cinnamaldehyde in a concentration dependent manner from 50 μM (control) to 220 μM (under 25 μM compound 18), suggesting that the two structural analogs compete for the same binding site but have opposite affects on channel activity (FIG. 3B). Compound 18 blocked cinnamaldehyde-induced TRPA1 currents in excised patches of Xenopus oocytes (FIG. 3C) and TRPA1 responses in CHO cells induced by cold or pressure (data not shown). To test the efficacy and specificity of compound 18 in vivo, we coinjected cinnamaldehyde and compound 18 in hindpaw of mice. 1-10 mM of compound 18 did not cause any behavioral response (data not shown). However, compound 18 significantly blocked cinnamaldehyde-induced but not capsaicin-induced nociceptive events, suggesting efficacy and specificity of this compound in blocking the nociception (FIG. 3D).

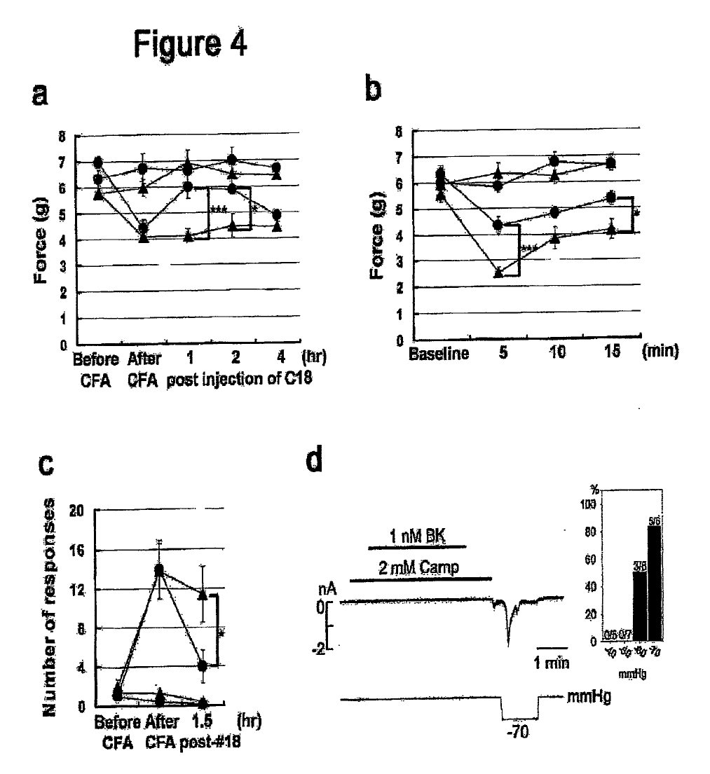

Hyperalgesia is defined as an increased response to painful stimuli (thermal and/or mechanical) due to injury or inflammation. We observed that nociceptive responses to acute heat or pressure to the paw were not affected by compound 18 (data not shown). However, compound 18 relieved mechanical hyperalgesia induced by complete Freund's adjuvant (CFA) injection into the hindpaw when injected 24 hours after CFA (FIG. 4A). A similar reduction in mechanical nociceptive behavior was observed with a short term hyperaglesia model (bradykinin injection) (FIG. 4B). Importantly, we found that compound 18 did not block CFA- or bradykinin (BK)-induced heat hyperalgesia (data not shown), providing additional evidence of specificity of the compound. These behavioral assays described here were also performed with a structurally-unrelated compound that blocks TRPA1 (Compound 40; N,N′-Bis-(2-hydroxybenzyl)-2,5-diamino-2,5-dimethylhexane), with very similar results. Together, these in vivo data indicate that blocking TRPA1 relieves mechanical-hyperalgesia, but not heat-hyperalgesia.

### Example 3

### Further Evidence of TRPA1 Function in Mechanical and Cold Hyperalgesia

In our hands, it is not possible to assay for noxious cold response in mice. For example, mice do not show nociceptive responses to cold temperatures as low as 0° C., and no cold-allodynia in response to CFA. Cold-activation of TRPA1 has been disputed, but an in vivo role in cold hyperalgesia in rats has been recently suggested (Jordt et al., Nature 427:260, 2004; and Obata et al., J Clin Invest 115:2393, 2005). We therefore used rats to address a role for TRPA1 using compound 18. We found that rat TRPA1 is also blocked by compound 18, similar to human and mouse TRPA1 (data not shown). We observed robust block of CFA-induced cold hyperalgesia in rats with compound 18 on a 5° C. plate (FIG. 4C). Collectively, the data suggests that TRPA1 is acting both as a cold receptor and mechanoreceptor in vivo, but only after sensitization by inflammatory or injury signals. Consistently, it was found that TRPV1 null mice show a strong thermal hyperalgesia phenotype, but they show no or mild phenotype in acute thermosensation (Davis et al., Nature 405:183, 2000, and Caterina et al., Science 288:306, 2000). A role for TRPA1 in mechanical hyperalgesia could be explained if TRPA1 is sensitized to respond to lesser mechanical threshold in response to inflammation. This is similar to modulation of heat sensitivity of TRPV1. TRPV1 normally has an activation threshold of 43° C., but a variety of inflammatory signals sensitize TRPV1 to activate at lower temperatures.

To test this possibility, we examined if BK signaling can reduce mechanical threshold of TRPA1. After a 3 minute pre-treatment with 1 nM BK pre-treatment for 3 min, CHO cells cotransfected with bradykinin B2 receptor and TRPA1 showed mechanical responses to −60 mmHg pressure stimulation (FIG. 4D). The sensitized response of TRPA1 provides a potential molecular mechanism for the physiological role of TRPA1 in mechanical hyperalgesia. In CHO cells, the response of TRPA1 to pressure is not instantaneous (with onset time varying in order of seconds), which suggests that TRPA1 is not directly activated by stretch, and is probably activated via a second message. Interestingly, BK application reduces the threshold of activation and curtails the delay.

### Example 4

### General Materials and Methods

Mammalian cell electrophysiology: ThermoTRP-expressing CHO cells (rat TRPV1, rat TRPV2, mouse TRPV3, rat TRPV4, mouse TRPM8, and mouse TRPA1), control CHO cells, and cultured rat DRG neurons were prepared as described in Story et al., Cell 112:819, 2003; and Bandell et al., Neuron 41:849, 2004. Electrophysiological recordings were performed as described in Bandell et al., Neuron 41:849, 2004. Briefly, CHO cells were clamped at −60 mV and 0.8 second ramps from −80 mV to +80 mV were run every 4 seconds. Currents from DRG neurons were recorded at −60 mV and for their current-voltage curve, 300 ms voltage step at +20 mV was used 40 ms before 800 ms ramp from -80 mV to +80 mV to minimize contamination of voltage gated Na⁺ or Ca²⁺ current. The pipette solution for temperature and hyperosmotic experiments consisted of (in mM) 140 CsCl, 5 EGTA, 10 HEPES, 2 MgATP, 0.2 NaGTP, titrated to pH 7.4 with CsOH. The base external solution for these experiments consisted of (in mM) 140 NaCl, 5KCl, 10 HEPES, 2CaCl₂, 1MgCl₂, titrated to pH 7.4 with NaOH. Mannitol was used to adjust osmolarity for hypertonic solutions. For mouse TRPV3 and rat TRPV2, external calcium was replaced with 5 mM EGTA. Gluconate was substituted for chloride in (±)-pressure and hypotonic experiments to eliminate the potential for endogenous swelling-activated chloride currents. For these experiments, pipette solution (295 mOsm) consisted of (in mM) 125 Cs-gluconate, 15CsCl, 5 EGTA, 10 HEPES, 2 MgATP, 0.2NaGTP, titrated to pH 7.4 with CsOH. The external solution consisted of (in mM) 90 Na-gluconate, 10 NaCl, 5 K-gluconate, 10 HEPES, 2 CaCl₂, 1 MgCl₂, titrated to pH 7.4 with NaOH. Osmolarity was adjusted with mannitol to 220 mOsm (hypotonic) or 298 mOsm 15 (isotonic). (±)-Pressure was hydrostatically delivered by the recording pipette using syringe pumping (Hamill et al., Annu Rev Physiol 59:621, 1997) and monitored through Pressure Monitor (World Precision Instruments). Warner temperature controller (TC-324B and CL-100) was used for the heating or cooling the perfused bath solutions. Experiments in which junction potentials/access resistances varied significantly or a standing current over -100 pA at −60 mV was developed without any stimulus were discarded. All thermoTRPs other than TRPA1 tested did not respond to mechanical stimuli. The number of cells (n) tested with -100 mmHg to −300 mmHg, ˜+100 mmHg, 450 mOsm, and 220 mOsm for each cell type, respectively, are: CHO cells, n=7, 14, 5, 12; TRPV1, n=6, 5, 7, 5; TRPV2, n=4, 5, 3, 5; TRPV3, n=3, 2, 3, 0; TRPM8, n=12, 4, 10, 0. TRPV3 and TRPM8 were known not to respond to hypotonic solutions.

FM1-43 experiments: The FM1-43 labeling of CHO cells transfected with mTRPA1 was performed as described (Meyers et al., J Neurosci 23:4054, 2003). Briefly, CHO cells were transfected using Fugene (Roche) with mTRPA1-pcDNA5. For mock transfection CHO cells were treated with Fugene, but without any plasmid DNA. 24 hrs after transfection the cells were incubated for 5 min with 200 μM cinnamaldehyde in physiological buffer (consisted of (in mM) 130 NaCl, 3 KCl, 2 MgCl₂, 2 CaCl₂, 10 HEPES, 10 glucose) at room temperature, followed by 3 min with 10 μM of FM1-43. The cells were then washed thoroughly and imaged. mTRPA1 and hTRPA1-expressing CHO cells were tested in whole cell patch clamp configuration using the PatchXpress (Axon Instruments) for the effect of the FM143 dye on TRPA1 activation. Cells were plated the day before testing and induced with 0.5 μg/ml tetracycline as previously described in Story et al., Cell 112:819, 2003. Immediately prior to testing, cells were trypsinized and resuspended in calcium-free DMEM media (Invitrogen). Recordings were performed in extracellular solution containing (in mM) 2.67 KCl, 1.47 KH2PO4, 0.5 MgCl₂, 138 NaCl, 8 Na2HPO4, 5.6 glucose. The intracellular solution contained (in mM) 140 KCl, 10 HEPES, 20 glucose, 10 HEDTA and 1 μM buffered free Calcium. Holding currents at −80 mV were used for quantitative analysis of TRPA1 activation and inhibition. Experiments involved an initial application of 100 μM cinnamaldehyde to elicit a current in cells followed by a second addition of cinnamaldehyde and 10 μM FM1-43. An inhibition of the current was observed in 7/8 cells expressing mTRPA1 and 3/4 cells expressing hTRPA1. On average, a 50% block in current was observed.

FLIPR Screen: CHO cells expressing human TRPA1 were plated into 384 well plates at a concentration of ˜8,000 cells/well. Cells were transferred to phosphate-buffered saline (PBS) and loaded with the calcium sensitive dye FLUO-4 using the FLIPR Calcium 3 Assay Kit (Molecular Devices, Sunnyvale, Calif.) 1 hour prior to the assay. Assays were run using the FLIPR2 (Molecular Devices, Sunnyvale, Calif.). All compounds were diluted into PBS from a high concentration DMSO-based stock solution and added during data collection with the FLIPR2 internal pipette head. Final DMSO concentrations never exceeded 0.5%.

Xenopus oocyte excised patches: Human TRPA1 was cloned into the pOX expression vector (Jegla et al., J Neurosci 17:32, 1997) and cRNA transcripts were produced using the T3 mMessage Machine kit (Ambion, Tex.). Mature 17 defolliculated Xenopus oocytes were injected with 50 nl of human TRPA1 cRNA at ˜1 μg/μl. Oocytes were incubated in ND96 (96 mM NaCl, 2 mM KCl, 1 mM MgCl2, 1.8 mM CaCl2, 5 mM HEPES, pH 7.4, supplemented with Na-pyruvate (2.5 mM), penicillin (100 u/ml) and streptomycin (100 μg/ml) 3-5 days to ensure expression. Vitelline envelopes were mechanically removed prior to recording. Recordings were made under voltage clamp from excised patches in the inside-out configuration at room temperature with 1-1.5 MΩ pipettes. The bath ground was isolated using an agar bridge. Capacitance and series resistance were compensated and solutions that eliminate native calcium-activated chloride currents were used (Patch electrode (in mM): 140 NaMES, 4 NaCl, 1 EGTA, 10 HEPES, pH 7.2; bath solution: 140 KMES, 4 KCl, 1 EGTA, 10 HEPES, pH 7.2). Compounds were added to the bath solution. Currents were recorded using a Multiclamp 700B amplifier and the pCLAMP acquisition suite.

Behavioral assays: Mice (C57B16 Mus musculus) of 8-10 weeks in age and 150-250 g Sprague Dawley rats were used for all behavioral assays. Animals were acclimated for 20-60 min to their testing environment prior to all experiments. Students' T test was used for all statistical calculations. All error bars represent standard error of the mean (SEM). Thermal plates, Hargreaves method (Plantar Analgesia meter) and Von Frey apparatus (Dynamic Plantar Aesthesiometer) were from UGO Basile and Columbus instruments. Mechanical or thennal hyperalgesia assays were performed as described in Morqrich et al., Science 307:1468, 2005; and Caterina et al., Science 288:306, 2000). Briefly, mice were acclimated for 60 min to their testing environment prior to all experiments. Baseline responses were measured first and then 10 nM BK was injected to the skin of left hindpaws. Von Frey threshold or paw withdrawal latency was measured at 5, 15 and 30 min post injection. 1 mM of compound 18 was sometimes coinjected to left hindpaws to test its analgesic effect. For CFA-induced hyperalgesia test, 5 μg CFA in 10 uL was injected into mice (Caterina et al., Science 288:306, 2000; and Cao et al., Nature 392:390, 1998) and 50 μg in 100 uL (1:1 emulsion of mineral oil and saline; Obata et al., J Clin Invest 115:2393, 2005) was injected into rats and in 24 hrs measurements were performed. Before the measurement, the animals were re-acclimated to the environment for 20-60 min. Different time points were used for experiments with CFA-injected animals (30 min, 1, 1½, 2 and 4 hr after compound 18 injection).

Compounds: All chemicals were purchased from Sigma-Aldrich unless otherwise described. Capsaicin was purchased from Fluka. Stock solutions for ruthenium red (10 mM) or gadolinium chloride (100 mM) were made using water and were diluted with test solutions before use.

It is understood that the examples and embodiments described herein are for illustrative purposes only and that various modifications or changes in light thereof will be suggested to persons skilled in the art and are to be included within the spirit and purview of this application and scope of the appended claims. Although any methods and materials similar or equivalent to those described herein can be used in the practice or testing of the present invention, the preferred methods and materials are described.

All publications, GenBank sequences, ATCC deposits, patents and patent applications cited herein are hereby expressly incorporated by reference in their entirety and for all purposes as if each is individually so denoted.

We claim:

1. A method for treating hyperalgesia in a subject, comprising administering to the subject a pharmaceutical composition that comprises an effective amount of a TRPA1 antagonist, wherein the TRPA1 antagonist specifically blocks TRPA1 activation, thereby suppressing or inhibiting noxious chemosensation, thermosensation, and mechanosensation in the subject.

2. The method of claim 1, wherein the TRPA1 antagonist does not block activation of one or more of the other thermoTRPs selected from the group consisting of TRPV1, TRPV2, TRPV3, TRPV4 and TRPM8.

3. The method of claim 1, wherein the TRPA1 antagonist is (Z)-4-(4-chlorophynyl)-3-methylbut-3-en-2-oxime.

4. The method of claim 1, wherein the TRPA1 antagonist is N,N′-Bis-(2-hydroxybenzyl)-2,5-diamino-2,5-dimethylhexane.

5. The method of claim 1, wherein the TRPA1 antagonist is a TRPA1 antagonist antibody.

6. The method of claim 1, wherein the subject suffers from an inflammatory condition or a neuropathic pain.

7. The method of claim 1, wherein the subject suffers from mechanical or thermal hyperalgesia.

8. The method of claim 1, wherein the subject is a human.

9. The method of claim 1, further comprising administering to the subject a second pain-reducing agent.

10. The method of claim 9, wherein the second pain-reducing agent is an analgesic agent selected from the group consisting of acetaminophen, ibuprofen and indomethacin and opioids.

11. The method of claim 9, wherein the second pain-reducing agent is an analgesic agent selected from the group consisting of morphine and moxonidine.

12. A method for identifying an agent that inhibits or suppresses noxious mechanosensation, comprising (a) contacting test compounds with a cell that expresses the transient receptor potential ion channel TRPA1, and (b) identifying a compound that inhibits a signaling activity of an activated TRPA1 in the cell in response to a mechanical stimulus; thereby identifying an agent that inhibits or suppresses noxious mechanosensation.

13. The method of claim 12, further comprising examining the identified compound for effect on activation or signaling activities of one or more thermoTRPs selected from the group consisting of TRPV1, TRPV2, TRPV3, TRPV4 and TRPM8.

14. The method of claim 12, wherein the identified compound suppresses or reduces the signaling activity of the activated TRPA1 ion channel relative to the signaling activity of the TRPA1 ion channel in the absence of the compound.

15. The method of claim 12, wherein the identified compound does not block activation of one or more thermoTRPs selected from the group consisting of TRPV1, TRPV2, TRPV3, TRPV4 and TRPM8.

16. The method of claim 12, wherein the activated TRPA1 ion channel is activated by a TRPA1 agonist selected from the group consisting of cinnamaldehyde, eugenol, gingerol, methyl salicylate, and allicin.

17. The method of claim 12, wherein the cell is a TRPA1-expressing CHO cell, a TRPA1-expressing Xenopus oocyte, or a cultured DRG neuron.

18. The method of claim 12, wherein the signaling activity is TRPA1-induced electric current across membrane of the cell or calcium influx into the cell.

19. The method of claim 12, wherein the mechanical stimulus is suction pressure or hyperosmotic stress.

20. A use of a TRPA1-specific inhibitor in the manufacture of a medicament for treating thermal or mechanical hyperalgesia in a subject, wherein the TRPA1-specific inhibitor is (Z)-4-(4-chlorophynyl)-3-methylbut-3-en-2-oxime or N,N′-Bis-(2-hydroxybenzyl)-2,5-diamino-2,5-dimethylhexane.
